(19) United States
(12) Patent Application Publication
(10) Pub. No.: US 2010/0249744 A1
(43) Pub. Date: Sep. 30, 2010

(54) FASTENING TAPE AND DISPOSABLE DIAPER HAVING THE SAME

(76) Inventor: Ikuo Akiyama, Tsurugi-cho (JP)

Correspondence Address: Tomoko Nakajima;Cermak Nakajima LLP, 127 S. Peyton Street, Suite 210, Alexandria, VA, 22314, US

(21) Appl. No.: 12/707,952

(22) Filed: Feb. 18, 2010

(30) Foreign Application Priority Data
Mar. 24, 2009 (JP) 2009-072537

(51) Int. Cl. A61F 13/62

(52) U.S. Cl. 604/391

(57) ABSTRACT

A fastening tape comprising a tab part formed at one end of a tape substrate, a fixing part formed at the other end of the tape substrate so as to be attached to a disposable diaper main body, a fastening part formed between the tab part and the fixing part, wherein an attachment is fixed to one surface of the tape substrate at the fastening part, the fastening part is folded at a boundary between the fastening part and the fixing part to form a first fold so that the attachment is inside the first fold, the attachment is joined to the fixing part of the tape substrate, the tab part is folded at a boundary between the tab part and the fastening part to form a second fold so that the attachment is outside the second fold. According to the fastening tape of the above, the tab part of the fastening tape is easily pinched and less likely to be joined to the attachment by folding the tab part, and hence handleability of the fastening tape is improved.

This application claims priority under 35 U.S.C. §119 to Japanese Patent Application No. 2009-072537, filed Mar. 24, 2009, which is incorporated by reference.

### FIELD OF THE INVENTION

The present invention relates to a fastening tape and a disposable diaper having the fastening tape.

### DESCRIPTION OF THE RELATED ART

Conventionally, there is known a disposable diaper which is provided with a fastening tape. As the fastening tape, for example, a hook-and-loop fastener which consists of a hook member and a loop member is used. For example, Japanese Laid-Open Patent Publication No. 2005-319139 (Patent Document 1) discloses, as a fastening tape attached to a disposable diaper, a fastening tape in which a hook member is fixed to one surface of a tape substrate made of a nonwoven fabric at one end of the tape substrate so that the nonwoven fabric is exposed at a tip of the one end of the fastening tape, and a non-joining part where the folded end is not joined is provided adjacent to the hook member.

Patent Document 1 discloses the fastening tape provided with the non-joining part where the tip (tab part) of the fastening tape is not joined to the hook member when the tip is folded on a side to which the hook member is fixed. However, Patent Document 1 does not disclose that the tip of the fastening tape is made less likely to be folded on the side to which the hook member is fixed. In the fastening tape disclosed in Patent Document 1, the tip made of a nonwoven fabric is likely to be contacted wholly to another part of the fastening tape or another member, and therefore, it may be difficult to pinch the tip of the fastening tape.

The present invention has been achieved in view of the above circumstances, and an object of the present invention is to provide: a fastening tape in which a tip (tab part) of the fastening tape is easily pinched and is less likely to be joined to an attachment such as a hook member which is fixed to the fastening tape; and a disposable diaper having the fastening tape.

### SUMMARY OF THE INVENTION

A fastening tape of the present invention which solves the above problems comprises a tab part formed at one end of a tape substrate, a fixing part formed at the other end of the tape substrate so as to be attached to a disposable diaper main body, a fastening part formed between the tab part and the fixing part, wherein an attachment is fixed to one surface of the tape substrate at the fastening part, the fastening part is folded at a boundary between the fastening part and the fixing part to form a first fold so that the attachment is inside the first fold, the attachment is joined to the fixing part of the tape substrate, the tab part is folded at a boundary between the tab part and the fastening part to form a second fold so that the attachment is outside the second fold.

In the fastening tape of the present invention, since the tab part is folded at the boundary between the tab part and the fastening part to form the second fold so that the attachment is outside the second fold, the tab part can be easily pinched though the fastening part is joined to the fixing part, and therefore, the folded fastening part can be easily unfolded. In addition, during use of the fastening tape, since the tab part is less likely to be folded on a side to which the attachment is fixed, a nonwoven fabric or the like of the tab part becomes less likely to be joined to the attachment. Therefore, a joining area of the attachment does not decrease, and hence joining property of the attachment is sustained appropriately during use of the fastening tape.

The attachment is preferably fixed to the one surface of the tape substrate only at the fastening part. When the attachment is fixed to only the fastening part, the attachment is tend to be provided in a planar manner, and the entire attachment is easily joined to an attachment-receiving part during use of the fastening tape. In addition, the attachment is less likely to be joined to another member unintentionally in a state where the attachment is joined to the fastening tape itself or the attachment-receiving part, and hence handleability of the fastening tape is improved.

The tape substrate is preferably made of a nonwoven fabric, and the attachment is preferably a hook fastener made of a plastic. When the tape substrate is made of a nonwoven fabric, the fold is easily formed at the boundary between the tab part and the fastening part by folding the tab part. As a result, the tab part is easily maintained in a folded state. When the attachment is the hook fastener made of a plastic, the nonwoven fabric of the tape substrate is easily folded by using an edge of the hook fastener as a guide.

The tape substrate is preferably made of an embossed nonwoven fabric having a mass per unit area in a range of from 50 g/m² to 100 g/m². By using such nonwoven fabric, the fastening tape tends to have a high-tension strength, the nonwoven fabric is easily folded to form the fold, and the tab part easily sustains in a folded condition.

A disposable diaper of the present invention comprises a disposable diaper main body having a front part, a back part and a crotch part positioned between the front part and the back part, and the fastening tape of the present invention is attached to a side end of the front or back part of the disposable diaper main body. In the disposable diaper of the present invention, since the fastening tape is pinched easily, it is easy to wear or remove the diaper. In addition, a joining position of the fastening tape is easily adjusted when the diaper is worn.

In the fastening tape of the present invention, the tab part of the fastening tape is easily pinched and less likely to be joined to the attachment by folding the tab part, and hence handleability of the fastening tape is improved.

### BRIEF DESCRIPTION OF THE DRAWINGS

FIG. 1 shows an example of a fastening tape of the present invention, FIG. 1(a) shows a plain view, FIG. 1(b) shows a view in which a fastening part in FIG. 1(a) is unfolded, FIG. 1(c) shows a cross sectional view along line A-A in FIG. 1(a), and FIG. 1(d) shows a cross sectional view along line B-B in FIG. 1(b).

FIG. 2 shows an alternative example of a fastening tape of the present invention.

FIG. 3 shows an example of a disposable diaper provided with a fastening tape of the present invention.

FIG. 4 shows a cross sectional view along line C-C in FIG. 3.

FIG. 5(a) shows a cross sectional view along line D-D in FIG. 3, and FIG. 5(b) shows a cross sectional view along line E-E in FIG. 3

### DESCRIPTION OF THE PREFERRED EMBODIMENTS

A fastening tape of the present invention comprises a tab part formed at one end of a tape substrate, a fixing part formed at the other end of the tape substrate so as to be attached to a disposable diaper main body, and a fastening part formed between the tab part and the fixing part.

The tab part is formed at one end of the tape substrate. The tab part may consist of the tape substrate, and may further have an attachment. The tab part is formed by folding the one end of the tape substrate.

The fixing part is formed at the other end of the tape substrate and is a part which is attached to a disposable diaper main body. The disposable diaper main body is, for example, a disposable diaper without the fastening tape. The fixing part is fixed to the disposable diaper main body, for example, by applying an adhesive agent, heat-sealing, or ultrasonic bonding. The fixing part is also a part to which an attachment at the fastening part is detachably joined by folding the fastening part at a boundary between the fastening part and the fixing part so that the attachment is located inside.

The fastening part is formed between the tab part and the fixing part and is a part to which the attachment is fixed. The attachment is fixed to one surface of the tape substrate.

The attachment is fixed to at least the fastening part, and may be further fixed to the tab part. When the attachment is further fixed to the tab part, the attachment is fixed to the fastening part and the tab part on same surface of the tape substrate. The attachment is preferably fixed to only the fastening part. It is noted that the attachment is not fixed to the fixing part.

Examples of the attachment include a hook member and a loop member of a hook-and-loop fastener; and an adhesive such as an adhesive tape and an adhesive layer; and the like. When a hook member of a hook-loop-fastener, that is hook fastener, is used as the attachment, the fixing part is preferably provided with a loop member or made of a material that serves as a loop member such as a nonwoven fabric, a woven fabric, a knitted fabric and the like. When an adhesive tape is used as the attachment, the fixing part is preferably made of a plastic film from which the adhesive tape can be peeled off. By forming the fixing part as described above, the attachment and the fixing part are easily disjoined from each other during use of the fastening tape.

The hook fastener is preferably used as the attachment. As the hook fastener, a member having hooks of, for example, an anchor shape, a hook shape, a mushroom shape or the like may be used.

The fastening part and the fixing part are delimited by folding the fastening part at the boundary between the fastening part and the fixing part when the attachment at the fastening part is joined to the fixing part of the tape substrate. The fastening part and the tab part are delimited by folding the tab part at a boundary between the fastening part and the tab part.

A shape of the fastening tape is not particularly limited. The shape of the fastening tape may be, for example, polygonal such as rectangular; oval, or the like, and an elongated shape is preferable. The fastening tape may be formed in such a manner that one end of the tape substrate at which the tab part is formed or the other end of the tape substrate at which the fixing part is formed has, for example, a straight linear or meandering outline. One end of the tape substrate at which the tab part is formed has preferably a meandering outline in view of manufacturing efficiency and easily pinching of the tab part. On the other hand, the other end of the tape substrate at which the fixing part is formed has preferably a straight linear outline.

Sizes of the tab part, the fastening part and the fixing part are not particularly limited; however, a length of the fastening part is preferably shorter than that of the fixing part. When the length of the fastening part is shorter than that of the fixing part, the attachment becomes less likely to join to the disposable diaper main body, and hence the disposable diaper main body becomes less likely to be damaged from the attachment. A length of the tab part is not particularly limited; however, it is preferable that the length of the tab part is shorter than that of the fastening part in view of easily handling. The length of the tab part is preferably equal to or shorter than half of the length of the fastening part. Here, the lengths of the tab part, the fastening part, and the fixing part mean lengths in a direction from the tab part to the fixing part.

The fastening part is folded at the boundary between the fastening part and the fixing part to form a first fold so that the attachment is inside the first fold, and the attachment is joined to the fixing part of the tape substrate. Thus, the fastening part is folded at the boundary between the fastening part and the fixing part so that the attachment is located inside the first fold of the folded fastening tape, and as a result, the attachment faces the fixing part. The attachment is joined to the tape substrate at the fixing part which the attachment faces. Prior to use of the fastening tape, the fastening part is detachably joined to the fixing part of the tape substrate. In the fastening tape of the present invention, since exposure of the attachment is suppressed prior to use, the attachment is less likely to be joined unintentionally to another member, resulting in excellent in handleability. In addition, the attachment becomes less likely to damage another member.

When the fastening tape is used, the attachment at the fastening part is exposed by disjoining the attachment to the tape substrate and unfolding the folded fastening part. In unfolding the folded fastening part, if the tab part has not been folded, the tab part may be contacted wholly to the tape substrate, and it may be difficult to pinch the tab part. However, in the fastening tape of the present invention, the tab part is folded at the boundary between the tab part and the fastening part to form a second fold so that the attachment is outside the second fold. Thus, the tab part is folded at the boundary between the tab part and the fastening part so that the attachment is located outside the second fold of the folded fastening tape. Therefore, the tab part can be easily pinched, and the folded fastening part can be easily unfolded. In addition, during use of the fastening tape, since the tab part is less likely to be folded on a side to which the attachment is fixed, a nonwoven fabric or the like of the tab part becomes less likely to be joined to the attachment. In this case, a joining area of the attachment does not decrease, and hence joining property of the attachment is sustained appropriately during use of the fastening tape.

The attachment only needs to be fixed to at least the fastening part, and may be further fixed to the tab part; however the attachment is preferably fixed to only the fastening part. When the attachment is fixed to only the fastening part, the attachment is tend to be provided in a planar manner. In this case, the entire attachment is easily joined to an attachment-receiving part during use of the fastening tape. In addition, when the attachment is further fixed to the tab part, the attachment at the tab part is exposed even in a state that the fastening part is joined to the fixing part of the tape substrate, and hence there is the possibility that the attachment is unintentionally joined to another member. In this case, handleability of the fastening tape is likely to deteriorate. Here, the attachment-receiving part means a part or a member to which the attachment of the fastening tape is joined when a disposable diaper with the fastening tape is worn.

The tape substrate may be made of a nonwoven fabric, a woven fabric, a knitted fabric, a plastic film, a laminate of a nonwoven fabric and a plastic film, or the like. Examples of the laminate include a laminate in which a sheet of a nonwoven fabric and a sheet of a plastic film are stacked, and a laminate in which a plastic film interposed between nonwoven fabrics.

One surface of the tape substrate to which the attachment is fixed is preferably made of a material capable of joining the attachment. This is because the one surface of the tape substrate having the attachment thereon is a surface to which the fastening part is joined when the fastening part is folded at the boundary between the fastening part and the fixing part so that the attachment is located inside the first fold. When the hook fastener is used as the attachment, the one surface of the tape substrate having the attachment thereon is preferably made of a nonwoven fabric, a woven fabric or a knitted fabric. When the adhesive is used as the attachment, the surface of the tape substrate having the attachment thereon is preferably made of a plastic film.

A combination of the attachment and the tape substrate is preferably the hook fastener and a nonwoven fabric, respectively. More preferably, the attachment is the hook fastener made of a plastic, and the tape substrate is made of a nonwoven fabric. When the tape substrate is made of a nonwoven fabric, the second fold is easily formed at the boundary between the tab part and the fastening part by folding the tab part. As a result, the tab part is easily maintained in a folded state. In addition, when the attachment is the hook fastener made of a plastic, the nonwoven fabric of the tape substrate is easily folded by using an edge of the hook fastener as a guide.

A nonwoven fabric used for the tape substrate is preferably a nonwoven fabric manufactured by a spunbonding method, an air-through method, a point bonding method, a melt blowing method, an airlaid method, a combination of these methods, or the like. Further, a nonwoven fabric manufactured by the spunbond method or a SMS method which is a combination of the spunbond method and the melt blowing method is preferable, and a nonwoven fabric manufactured by the spunbond method is especially preferable. By using such nonwoven fabrics, the tape substrate with high strength is obtained easily.

A material of the nonwoven fabric used for the tape substrate can be selected as appropriate from synthetic fibers such as polypropylene, polyethylene, polyester and polyamide; natural fibers such as pulp and silk. Among them, synthetic fibers such as polypropylene, polyethylene and polyester are preferable; a polypropylene fiber or a polyester fiber is more preferable. When a nonwoven fabric obtained from such a material is used, the tape substrate with high strength is obtained easily.

The nonwoven fabric used for the tape substrate has preferably a mass per unit area of 50 g/m² or more, more preferably 55 g/m² or more, preferably 100 g/m² or less, and more preferably 85 g/m² or less. When the nonwoven fabric has a mass per unit area of 50 g/m² or more, the nonwoven fabric tends to have a sufficient strength that is not torn even if it is pulled, and the tab part easily sustains in a folded condition. When the nonwoven fabric has a mass per unit area of 100 g/m² or less, the nonwoven fabric is easily folded to form the fold.

The nonwoven fabric used for the tape substrate is preferably embossed. The embossed nonwoven fabric is partially compressed to have an enhanced rigidity, and hence the fold is easily formed.

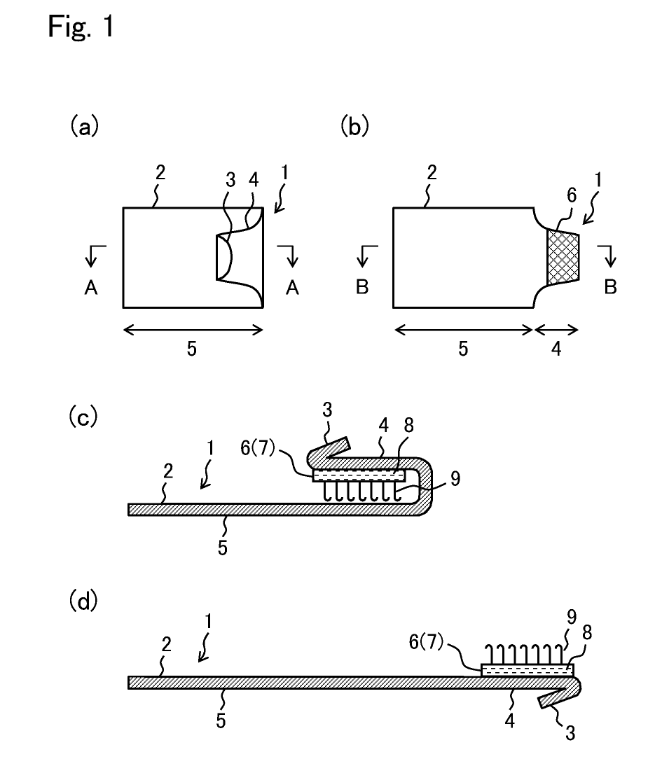

An example of the fastening tape of the present invention is explained in the following, referring to FIGS. 1 and 2.

FIG. 1(a) shows an example of the fastening tape of the present invention, and FIG. 1(c) shows a cross sectional view along line A-A of FIG. 1(a). A fastening tape 1 comprises a tab part 3 formed at one end of a tape substrate 2, a fixing part 5 formed at the other end of the tape substrate 2, and a fastening part 4 formed between the tab part 3 and the fixing part 5. FIG. 1(b) shows a spread state, that is an unfolded state of the folded fastening part 4, of the fastening tape 1 shown in FIG. 1(a), and FIG. 1(d) shows a cross sectional view along line B-B of FIG. 1(b).

An attachment 6 is fixed to one surface of the tape substrate 2 at the fastening part 4. In FIG. 1, a hook fastener 7, which comprises a base part 8 and a hook part 9, is provided as the attachment 6. The hook part 9 is composed of a plurality of hooks projected from one surface of the base part 8. The opposite surface of the base part 8, that is the opposite surface of the one surface with the hook part 9, is joined to the tape substrate 2. The fastening part 4 is folded at a boundary between the fastening part 4 and the fixing part 5 to form a first fold so that the attachment 6 is inside the first fold, and the attachment 6 is joined to the fixing part 5 of the tape substrate 2.

The tab part 3 is folded at a boundary between the tab part 3 and the fastening part 4 to form a second fold so that the attachment 6 is outside the second fold. Thus, the tab part 3 is folded at the boundary between the tab part 3 and the fastening part 4 on the side opposite to the attachment 6. As a result, even when the attachment 6 at the fastening part 4 is joined to the fixing part 5 of the tape substrate 2 as shown in FIGS. 1(a) and 1(c), the tab part 3 is easily pinched because of the folded tab part 3 that is folded on the side opposite to the attachment 6, resulting in easily unfolding the folded fastening part 4. In FIG. 1, the tab part 3 is shown to be folded nearly 180°, however, the tab part 3 is not necessarily folded to the extent degree shown in FIG. 1. Even when the tab part 3 is folded at an angle close to 0°, it becomes easy to pinch the tab part 3 as long as the tab part 3 is folded so that an end of the tab part 3 is lifted even slightly, and hence there is no problem.

FIG. 2(a) shows an alternative example of the fastening tape of the present invention. FIG. 2(a) shows a folded state that the fastening part 4 is folded to join to the fixing part 5, and FIG. 2(b) shows a spread state that the fastening part 4 is unfolded. In FIG. 2, one end of the tape substrate 2 where the tab part 3 is formed has a meandering outline with more meandering numbers than in FIG. 1, and the tab part 3 is folded. In the fastening tape shown in FIG. 2, since the attachments 6 are fixed on two sites, a wearer easily adjusts a size around his or her waist and trunk so as to fit a body shape of the wearer in wearing a diaper.

Next, a disposable diaper provided with the fastening tape of the present invention is explained. The disposable diaper of the present invention comprises a disposable diaper main body having a front part, a back part and a crotch part positioned between the front part and the back part, and the fastening tape is attached to a side end of the front or back part of the disposable diaper main body. Here, a part applied to an abdomen side of a wearer is called the front part, a part applied to a buttocks side of the wearer is called the back part, and a part positioned between the front part and the back part and applied to a crotch of the wearer is called the crotch part, in a state of wearing the disposable diaper.

The disposable diaper main body may comprise, for example, a laminate including a liquid-permeable top sheet, a liquid-impermeable back sheet and an absorbent core interposed therebetween, wherein the laminate has a front part, a back part and a crotch part therebetween. Also, the disposable diaper main body may comprise an outer sheet having a front part, a back part and a crotch part therebetween; and an absorbent main body including a liquid-permeable top sheet, a liquid-impermeable back sheet and an absorbent core interposed therebetween, wherein the absorbent main body is disposed on an inner surface, that faces a wearer in wearing, of the outer sheet at the crotch part.

The fastening tape is placed to the disposable diaper by joining the fixing part of the tape substrate to the disposable diaper main body. The fixing part of the tape substrate may be attached, for example, between two components constituting the disposable diaper main body, or on a surface of a component constituting the disposable diaper main body. The fixing part of the tape substrate is attached, for example, between the top sheet and the back sheet, between a side sheet and the back sheet, or on one surface of the outer sheet.

The fastening tape is preferably attached to the disposable diaper main body so that the attachment is not joined to the disposable diaper main body when the fastening part is folded at the boundary between the fastening part and the fixing part so that the attachment is located inside the first fold. Therefore, a length of the fixing part and a length by which the fixing part is attached to the disposable diaper main body are preferably adjusted as appropriate in accordance with a manner where the fastening tape is attached to the disposable diaper main body.

The fastening tape is attached to at least one part selected from the group consisting of a left side end of the front part, a right side end of the front part, a left side end of the back part, and a right side end of the back part. Preferably, a pair of the fastening tapes are attached to the left and right side ends of the front part, or the left and right side ends of the back part.

The disposable diaper main body is preferably provided with the attachment-receiving part. The attachment-receiving part is a part or a member to which the attachment of the fastening tape can be joined detachably. For example, when the fastening tape is attached to the front part, the attachment-receiving part is preferably disposed at the back part of the disposable diaper main body. Also, when the fastening tape is attached to the back part, the attachment-receiving part is preferably disposed at the front part of the disposable diaper main body.

The fastening tape is preferably attached to the disposable diaper main body so that the surface with the attachment faces a wearer. In this case, the attachment-receiving part is preferably disposed on an outer surface, that is an opposite surface of the surface facing a wearer in wearing, of the disposable diaper main body.

Thus, in the present invention, a pair of fastening tapes are preferably attached to the left and right side ends of the front or back part of the disposable diaper main body so that the surface with the attachment faces a wearer, and the attachment-receiving part is preferably disposed on the outer surface of the back or front part of the disposable diaper main body. In wearing the disposable diaper, the fastening part of the fastening tape is spread out, and then, the fastening tape attached to the back part is joined to the attachment-receiving part disposed at the front part, or the fastening tape attached to the front part is joined to the attachment-receiving part disposed at the back part, for example.

In the disposable diaper of the present invention, since the fastening tape is pinched easily, it is easy to wear or remove the diaper. For example, when the diaper is worn, it is easy to pinch the tab part of the fastening tape which is joined to the attachment-receiving part. Therefore, when the diaper is removed, it is easy to disjoin the fastening tape from the attachment-receiving part. In addition, when the diaper is worn, a joining position of the fastening tape is easily adjusted by disjoining the fastening tape from the attachment-receiving part and rejoining the fastening part to the attachment-receiving part.

Next, a material of each member of the disposable diaper of the present invention is explained. The top sheet is preferably composed of a liquid-permeable nonwoven fabric or the like, and the back sheet and the outer sheet are preferably composed of a liquid-impermeable plastic film, a water-repellent nonwoven fabric or the like. The outer sheet may be also composed of a laminate in which a hydrophilic nonwoven fabric and a water-repellent material such as a liquid-impermeable plastic film and a water-repellent nonwoven fabric are stacked.

In the case that the top sheet and the back sheet are composed of nonwoven fabrics, a mass per unit area of the nonwoven fabric used for the top sheet and the back sheet is preferably less than that of the nonwoven fabric used for the fastening tape. The nonwoven fabric used for the top sheet and the back sheet may have a mass per unit area of, for example, 5 g/m² or more and 35 g/m² or less.

The absorbent core can be obtained, for example, by the steps of mixing a hydrophilic fiber assembly such as crushed pulp fibers, cellulose fibers and the like with a granular absorbent resin to obtain a clump; wrapping the clump with a paper sheet such as a tissue paper and the like, or with a cover sheet such as a liquid-permeable nonwoven fabric sheet and the like; and molding the obtained wrapped clump into a predefined shape such as a rectangular shape, an hourglass shape, a center nipped-in gourd shape, a battledore shape and the like.

The attachment-receiving part is preferably composed of a material to which the attachment can be joined. When the adhesive is used as the attachment, the attachment-receiving part is preferably composed of a plastic film. When the hook fastener is used as the attachment, the attachment-receiving part is preferably composed of a loop member. The loop member may be composed of a nonwoven fabric, a woven fabric, a knitted fabric, a composite material of a plastic film having a nonwoven fabric or a woven fabric on a surface, or the like. The each material used as the loop member preferably has a loop structure on a surface thereof. In addition, when the attachment-receiving part is disposed on the outer surface of the disposable diaper main body and the back sheet or the outer sheet is composed of a material that serves as the attachment-receiving part, another member which functions as the attachment-receiving part may not be employed.

Examples of a bonding means for bonding various members constituting the disposable diaper include an adhesive agent, heat sealing, ultrasonic sealing, and the like. Examples of the adhesive agent include a hot-melt adhesive such as a polyolefin adhesive, a rubber adhesive, a vinyl acetate adhesive, and the like.

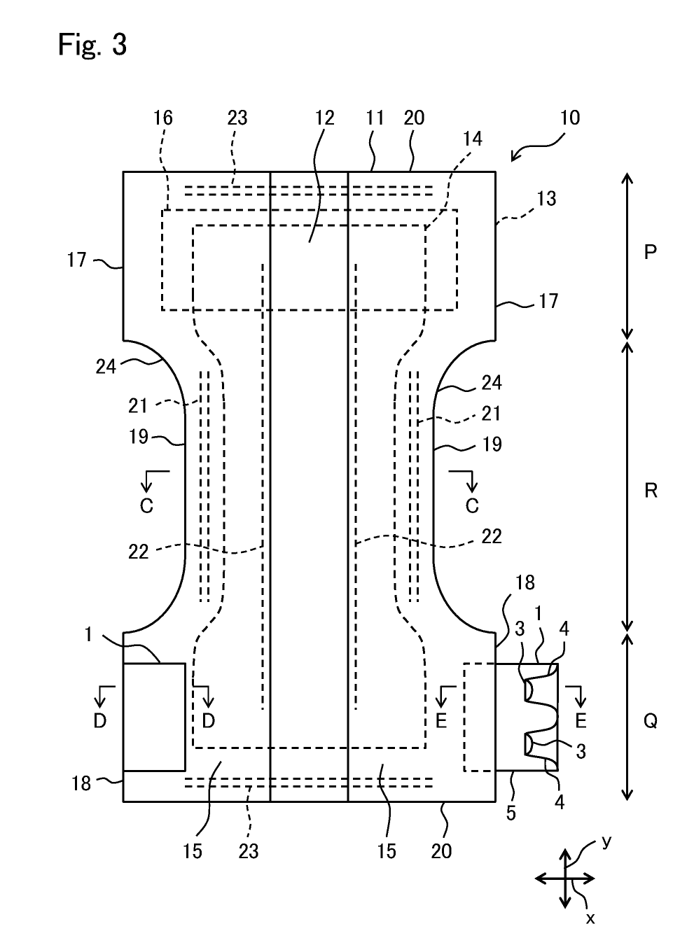

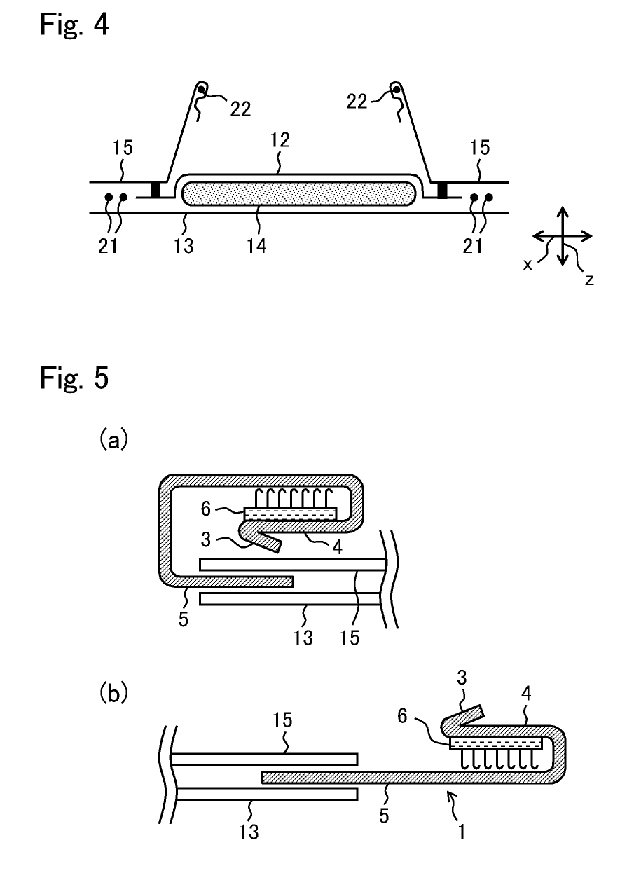

An example of the disposable diaper of the present invention is explained, referring to FIGS. 3 to 5. FIG. 3 shows an example of the disposable diaper of the present invention, FIG. 4 shows a cross sectional view along line C-C of FIG. 3, FIG. 5(a) shows a cross sectional view along line D-D of FIG. 3, and FIG. 5(b) shows a cross sectional view along line E-E of FIG. 3. In FIGS. 3 and 4, the arrow x direction is defined as a lateral direction (a width direction) and the arrow y direction is defined as a longitudinal direction (a lengthwise direction). A vertical direction with regard to a plane formed by the arrows x and y is defined as a vertical direction z (a thickness direction).

A disposable diaper 10 comprises a disposable diaper main body 11 having a front part P, a back part Q and a crotch part R therebetween. Leg cutouts 24 are formed at the crotch part R so that a wearer easily steps into the diaper.

The disposable diaper main body 11 comprises a laminate including a liquid-permeable top sheet 12, a liquid-impermeable back sheet 13 and an absorbent core 14 interposed therebetween, and the laminate has the front part P, the back part Q and the crotch part R therebetween. The top sheet 12 is placed so as to face a wearer's skin, and allows excrement such as urine and the like to permeate through. The excrement that permeated the top sheet 12 is accommodated in the absorbent core 14. The back sheet 13 prevents the excrement from leaking outside, thereby protecting clothes and the like becoming soiled.

In FIG. 4, side sheets 15, which extend in the longitudinal direction y, are provided on left and right side ends, with respect to the lateral direction x of the disposable diaper 10, of the top sheet 12. The side sheet 15 is joined to an outer end of the top sheet 12. An erection elastic member 22 is disposed at an inner end of the side sheet 15. When the disposable diaper 10 is worn, an inner part of the side sheet 15 rises above the top sheet 12 due to a shrinkage force of the erection elastic member 22. Thus, the inner part of the side sheet 15 rises toward a wearer's skin, thereby preventing excrement such as urine and the like from leaking outward in the lateral direction x. The side sheet 15 is preferably composed of a liquid-impermeable plastic film, a water-repellent nonwoven fabric or the like, and more preferably composed of a water-repellent nonwoven fabric. The nonwoven fabric used for the side sheet preferably has a mass per unit area of 5 g/m² or more and 35 g/m² or less.

Leg elastic members 21 are disposed between the side sheet 15 and the back sheet 13 in a stretched state along left and right side edges 19 of the crotch part R of the disposable diaper 10. Leg-gathers around wearer's legs are formed due to a shrinkage force of the leg elastic members 21, thereby preventing excrement such as urine and the like from leaking from the crotch part.

A waist elastic member 23 is disposed between the top sheet 12 or the side sheet 15 and the back sheet 13 in a stretched state along an edge 20 in the longitudinal direction y of the disposable diaper 10. A waist-gather around a wearer's waist is formed due a shrinkage force of the waist elastic member 23, thereby preventing excrement such as urine and the like from leaking from a back side or an abdomen side.

A pair of fastening tapes 1 are attached to left and right side ends 18 of the back part Q of the disposable diaper main body 11. In FIG. 5, the fastening tape 1 is attached between the side sheet 15 and the back sheet 13 at the fixing part 5 so as to extend outward in the lateral direction x from the disposable diaper 10. An attachment-receiving part 16 is disposed at the front part P. Although it is not shown in FIG. 3, the fastening tapes 1 may be attached to left and right side ends 17 of the front part P, and the attachment-receiving part 16 may be disposed at the back part Q.

FIG. 5(a) shows a D-D cross-sectional view of the fastening tape 1 which is attached to the left side end 18 of the back part Q in FIG. 3 (which is located on a left side in the drawing and on a left side of a wearer when the diaper is worn). In the fastening tape 1 shown in FIG. 5(a), the fastening part 4 is folded so that the attachment 6 at the fastening part 4 is joined to the fixing part 5, and further the fixing part 5 is folded along a side edge of the disposable diaper main body 11, whereby the fastening tape 1 is folded back on a surface of the disposable diaper main body 11. The fastening tape 1 attached to the disposable diaper 10 which is in an unused state that it is just manufactured is preferably in the state shown in FIG. 5(a), because the disposable diaper 10 becomes compact.

FIG. 5(b) shows an E-E cross-sectional view of the fastening tape 1 which is attached to the right side end 18 of the back part Q in FIG. 3 (which is located on a right side in the drawing and on a right side of a wearer when the diaper is worn). FIG. 5(b) shows a state that the folded fixing part 5 of the fastening tape 1 in FIG. 5(a) is unfolded. When the diaper is worn, the fastening tape 1 is unfolded from the state in FIG. 5(a) to the state in FIG. 5(b), and further the folded fastening part 4 is unfolded, whereby the fastening tape becomes ready for use.

1. A fastening tape comprising
a tab part formed at one end of a tape substrate,
a fixing part formed at the other end of the tape substrate so as to be attached to a disposable diaper main body,
a fastening part formed between the tab part and the fixing part, wherein
an attachment is fixed to one surface of the tape substrate at the fastening part,
the fastening part is folded at a boundary between the fastening part and the fixing part to form a first fold so that the attachment is inside the first fold,
the attachment is joined to the fixing part of the tape substrate,
the tab part is folded at a boundary between the tab part and the fastening part to form a second fold so that the attachment is outside the second fold.

2. The fastening tape according to claim 1, wherein the attachment is fixed to the one surface of the tape substrate only at the fastening part.

3. The fastening tape according to claim 1, wherein the tape substrate is made of a nonwoven fabric, and the attachment is a hook fastener made of a plastic.

4. The fastening tape according to claim 1, wherein the tape substrate is made of an embossed nonwoven fabric having a mass per unit area in a range of from 50 g/m² to 100 g/m².

5. A disposable diaper comprising the fastening tape according to claim 1, wherein
the disposable diaper comprises a disposable diaper main body having a front part, a back part and a crotch part positioned between the front part and the back part, and
the fastening tape is attached to a side end of the front or back part of the disposable diaper main body.
